(19) United States
(12) Patent Application Publication
(10) Pub. No.: US 2018/0028114 A1
(43) Pub. Date: Feb. 1, 2018

(54) SYSTEM AND METHOD FOR PROVIDING PLACEBO INFORMATION TO A USER OF A WEARABLE DEVICE

(71) Applicant: KONINKLIJKE PHILIPS N.V., EINDHOVEN (NL)

(72) Inventor: JOHN CRONIN, BONITA SPRINGS, FL (US)

(21) Appl. No.: 15/547,900

(22) PCT Filed: Feb. 3, 2016
(86) PCT No.: PCT/EP2016/052213

§ 371 Date: Aug. 1, 2017

Related U.S. Application Data
(60) Provisional application No. 62/113,710, filed on Feb. 9, 2015.

(30) Foreign Application Priority Data
Jul. 14, 2015 (EP) 15176656.5

(51) Int. Cl. A61B 5/00; G06F 19/00

(52) U.S. Cl. CPC A61B 5/4848; G06F 19/3406; G06F 19/322; G06F 19/345; A61B 5/6801; A61B 5/0022; A61B 5/742; G06F 17/30979

(57) ABSTRACT

Various embodiments described herein generally relate to a wearable device collecting data from one or more sensors related to the activity of a person and the wearable device communicating the physiological data sensed or information that corresponds to the physiological data sensed to a recommendation network. In certain instances the recommendation network may analyze the physiological data or information that corresponds to the physiological data when composing messages. The messages composed by the recommendation network may include information that is skewed, biased, or otherwise altered from actual data sensed at a wearable device.

### TECHNICAL FIELD

Various embodiments described herein generally relate to a wearable device that collects sensor data from one or more sensors. More specifically, but not exclusively, various embodiments relate to a wearable device configured for altering the value of the sensed one or more types of physiological data to a predefined value while keeping the types of physiological data unchanged.

### BACKGROUND

Wearable technology may include any type of mobile electronic device that can be worn on the body, attached to or embedded in clothes and accessories of an individual. Such wearable technology has been used in a variety of areas, including monitoring vital signs and other health data of the user as well as other types of data. Examples of some wearable technology in the health arena include Fitbit®, Nike+® FuelBand®, and the Apple Watch® devices.

Doctors are well aware that patients report benefits after receiving a treatment or “pharmaceutical” even when the treatment or “pharmaceutical” is a placebo (i.e., not a real treatment or pharmaceutical). Many patients after receiving a placebo report benefits that may include reduced pain or reductions in blood pressure even when the placebo is known not to produce a pharmacological response in a person. In some cases, beneficial effects are reported even when the patient knows that they received a placebo.

### SUMMARY

It is therefore an object of various embodiments to provide a device and a method for providing messages to a wearable device that include placebo information.

According to various embodiments a computer-implemented method for altering sensed physiological data derived from a user of a wearable device is provided. The method comprises receiving one or more types of physiological data of the user sensed by one or more corresponding sensors in the wearable device; altering the value of the one or more types of physiological data to a first predefined value based on a predetermined altering rule database while keeping the types of physiological data unchanged; notifying the first altered physiological data to the user.

In some embodiments, the altered physiological data is an inaccurate physiological data with respect to the physiological data sensed by the corresponding sensors in the wearable device.

In some embodiments, the altered physiological data is placebo data.

In some embodiments, the computer-implemented method further comprises detecting the reaction of the user in response to the first altered sensed physiological data; and providing different types of placebo information to the user based on the data structure table.

In some embodiments, the computer-implemented method further comprises detecting the reaction of the user in response to the first altered sensed physiological data; and providing different types of placebo information to the user in response to the detection result.

In some embodiments, the detecting the reaction of the user in response to the first altered sensed physiological data further comprising the steps of receiving one or more types of physiological data of the user sensed by one or more corresponding sensors in the wearable device after a predefined duration that the first altered physiological data is provided to the user; comparing the difference of the received physiological data of the user and the first altered physiological data is provided to the user.

In some embodiments, the different types of placebo information comprise at least one of: a second altered physiological data; and additional encouraging information.

In some embodiments, the computer-implemented method further comprises communicating with a medicine administration system by indicating whether the placebo medicine needs to be provided to the user, or whether an increase of the amount of placebo medicine to the current user.

In some embodiments, the predetermined altering rule database is stored in a recommendation network.

In some embodiments, the computer-implemented method further comprises transmitting the physiological data sensed by the one or more sensors to a recommendation network; receiving an alternated physiological data from the recommendation network; and notifying the received alternated physiological data to the user.

In some embodiments, the computer-implemented method further comprises receiving a selection of a professional that may receive the physiological data sensed at the wearable device.

In some embodiments, the computer-implemented method further comprises receiving an indication allowing the professional to modify the physiological data sensed by the one or more corresponding sensors, wherein a modification by the professional skews physiological data sensed by the one or more corresponding sensors, the modified physiological data corresponds to the predetermined physiological data.

According to various embodiments, a wearable device is provided. The wearable device comprises a communication interface configured to receive data from at least one other device and to send a notification to a user of the wearable device or information to at least the one other device, a processor configured to perform a method as mentioned above. In some embodiments, the wearable device comprises a memory configured to store a predetermined altering rule database.

According to various embodiments, an altering rule engine is provided. The altering rule engine comprises a communication interface configured to communicate with at least one other device and to send a notification to the user of the wearable device or information to at least the one other device, and a processor configured to perform a method as mentioned above.

According to various embodiments, a non-transitory computer readable data storage medium having embodied thereon a program executed by a processor to perform a method as mentioned above is provided.

Various embodiments described herein are based on the incentive that by merely providing an altered physiological data to the user without changing the type of the physiological data, a more believable motivational message can be provided to the user in an easier and simplified manner way by using very limited processing resource in the wearable device. Advantageously, the anxiety levels of a user wearing the wearable device are reduced. In addition, various embodiments may provide further motivational message that is skewed or biased in a direction that makes a health condition appear to be improving faster than it really is.

### BRIEF DESCRIPTION OF THE DRAWINGS

FIG. 1 illustrates a wearable device communicating with a recommendation network and with a mobile device according to an embodiment of the present invention.

FIG. 2 illustrates an exemplary wearable GUI that may be displayed on the display of the wearable device according to an embodiment of the present invention.

FIG. 3 illustrates an exemplary recommendation network base software that may execute on a recommendation network according to an embodiment of the present invention.

FIG. 4 illustrates a table that cross-references data that may be stored in a wearable database according to an embodiment of the present invention.

FIG. 5 illustrates an exemplary implementation of wearable application software and wearable software running on a wearable device according to an embodiment of the present invention.

FIG. 6 illustrates a mobile device architecture that may be utilized to implement the various features and processes described herein according to an embodiment of the present invention.

FIG. 7 illustrates an exemplary method that may be implemented according to an embodiment of the invention.

FIG. 8 illustrates an exemplary implementation of the wearable device according to an aspect of the invention.

FIG. 9 illustrates a data structure table that may be stored in an altering rule database according to an embodiment of the present invention.

FIG. 10 illustrates an exemplary method that may be implemented according to an embodiment of the invention.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

Various embodiments described herein generally relate to a wearable device collecting physiological data from one or more sensors at a wearable device. The wearable device may communicate the physiological data sensed or information that is derived from the physiological data sensed to a recommendation network. In some instances the recommendation network may analyze the physiological data or the derived information when composing messages. The messages composed by the recommendation network may include information that is skewed, biased, or otherwise altered in a positive direction. For example, a lowest blood pressure reading of a plurality of blood pressure readings may be reported in a motivational message even when the reported blood pressure reading is below an average or median blood pressure when compared to the plurality of blood pressure readings. In another instance, the blood pressure reading reported may be lower than a measured blood pressure that was measured by a sensor at the wearable device. The messages sent to the wearable device or to a mobile device of a user may include placebo information that is biased, skewed, or otherwise altered as compared to actual measurements made by sensors at a wearable device. Alternatively, in some embodiments, such bias, skew, or other alterations may be performed by a device other than a recommendation network such as, for example, the wearable device itself. Embodiments of the present invention may provide placebo information when attempting to help the user of a wearable device relax or stop obsessing over a health condition. The present invention may reduce anxiety levels of a user wearing the wearable device.

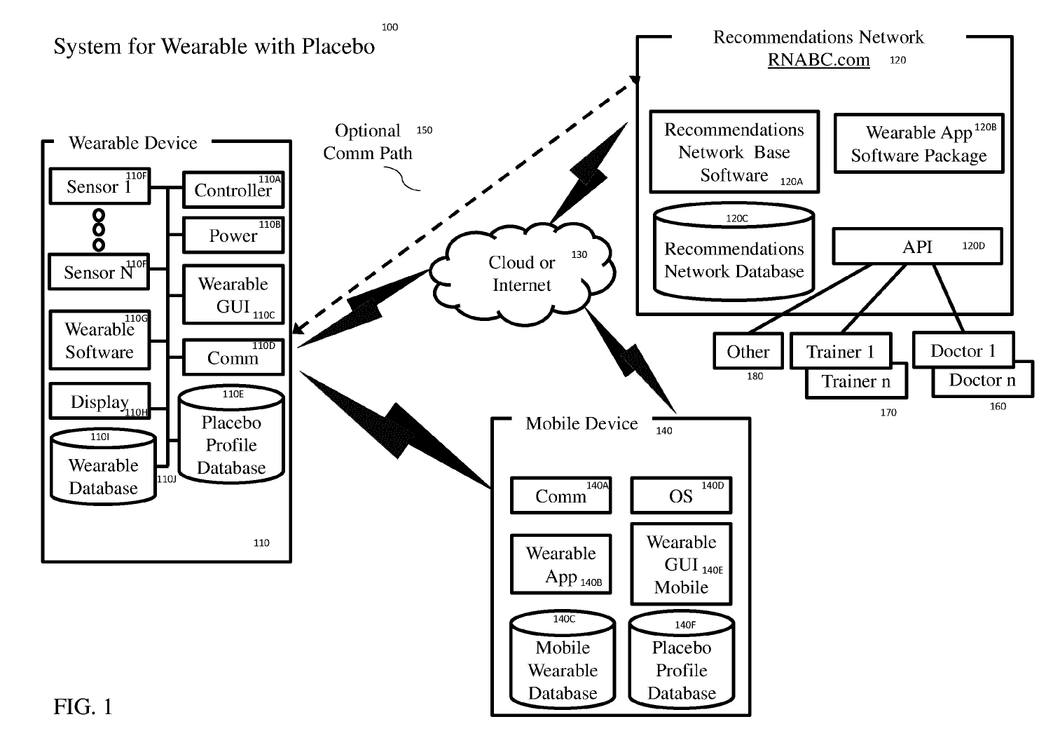

Sensors at a wearable device consistent with the embodiments described herein may include sensors useful in sensor physiological data of the user such as, for example, accelerometers, conductance sensors, optical sensors, temperature sensors, microphones, cameras, etc. The raw data provided by such sensors may be further processed by the wearable device or other devices to extract additional physiological parameters descriptive of the wearer such as, for example, steps taken, walking/running distance, standing hours, heart rate, respiratory rate, blood pressure, sweat pH, perspiration rate, stress level, body temperature, calories burned, resting energy expenditure, active energy expenditure, height, weight, sleep metrics, an amount ultraviolet radiation received, etc. FIG. 1 illustrates a wearable device communicating with a recommendation network and with a mobile device. The wearable device 110 may communicate with the recommendation network 120 over a data network, labeled as cloud or Internet 130 and may communicate with the mobile device 140 using any wireless data transmission technology standard in the art. While the example data network 130 is illustrated as a cloud network or the Internet, it will be apparent that in various alternative embodiments, additional or alternative data networks may be used such as, for example, carrier networks (e.g., 3G/LTE/4G/etc.) or LANs. The mobile device 140 may also communicate with the recommendation network 120 or the wearable device 110 over the cloud or Internet 130.

FIG. 1 illustrates a recommendation network 120 communicating with the wearable device 110 over an optional data communication path 150. As used herein, while the recommendation network 120 may include a network of devices (e.g., servers, blades, virtual machines, infrastructure devices, etc.), the recommendation network 120 may also constitute only a single device (e.g., a server, blade, or VM) for performing the functions described herein in association with the recommendation network 120. The wearable device 110 includes a controller 110A, a power supply 110B, a wearable graphical user interface (GUI) 110C, a communication interface 110D, a placebo profile database 110E, sensors 1 through N 110F, wearable software 110G, a display 110H, a wearable database 110I, and a bus 110J that interconnects various elements in the wearable device 110.

The mobile device 110 in FIG. 1 includes a communication interface 140A, a wearable application (APP) 140B, a mobile wearable database 140C, operating system software (OS) 140D, a wearable mobile GUI 140E, and a placebo profile database 140F. The recommendation network 120 includes recommendation network base software 120A, a wearable application (APP) software package 120B, a recommendation network database 120C, and an application program interface (API) 120D. The API in the recommendation network 120 may be used by one or more doctors 160, one or more trainers 170, or one or more other individuals 180 when interacting with the recommendation network.

As will be understood, to the extent that various embodiments are described herein with respect to software (e.g., wearable app 140B, OS 140D, etc.) “performing” various functionalities, such functionalities are actually performed by hardware, such as a microprocessor executing the software. As used herein, the term “processor” will be understood to encompass hardware such as microprocessors, field programmable gate arrays (FPGAs), application-specific integrated circuits (ASICs), or any other device capable of processing information according to the various embodiments described herein. Further, in embodiments utilizing ASICs for one or more functions described herein, the “software” defining such functionality may be omitted where the functionality is hard-wired into the design of the ASIC. As used herein, the term “memory” will be understood to encompass any device capable of storing data including L1/L2/L3 cache (e.g. SRAM), system memory (e.g., DRAM), and storage (e.g., flash memory, magnetic storage, optical storage). As used herein, the term “non-transitory machine-readable storage medium” will be understood to encompass both volatile and non-volatile memories, but to exclude transitory signals.

Placebo information provided to the wearable device 110 or a user mobile device 140 may be input or adjusted by a doctor 160 or trainer 170 that accesses the recommendation network 120. The doctor 160 or trainer 170 may enter the placebo information over API at the recommendation network 120. In certain instances other the doctors 160 or trainers 170 may communicate with the recommendation network 120 over the cloud or Internet 130.

A doctor 160 or trainer 170 interacting with the API may allow the doctor 160 or trainer 170 to modify or adjust information that may be sent in a message to a wearable device or a user mobile device. The information modified or adjusted may be placebo information that skews, biases, or otherwise alters health information sent to the wearable device or the user mobile device according to guidelines from the doctor 160 or trainer 170. Sensors 1-N 110F may sense data that corresponds to one or more physiological metrics. Sensors 1-N 110F may sense data related to the blood pressure, the body temperature, a heart rate, a number of calories burned, or data related to other physiological metrics of a user of a wearable device. Sensed data may be stored in the placebo profile database 110E at the wearable device 110. Recommendations may be downloaded from the recommendations network 120 and stored in the wearable database 110I at the wearable device 110. Information in the downloaded recommendations may have been entered by a doctor 170, a trainer 180, or by another individual 180. Over time, the wearable device may store sensor data in the profile database 110E and the wearable software 110G executing on a processor out of memory may compare sensor data with a recommendation when preparing to display a recommendation on display 110H when the sensor data sensed matches information in a recommendation. When the recommendation information identifies that information displayed on display 110H should include placebo information that skews, biases, or otherwise alters data sensed by a sensor at the wearable device 110, a message including the placebo information may be displayed on the display 110H. For example, when sensed data indicates that a user blood pressure has varied between 110 and 120 and placebo information indicates that a placebo rate of 90 should be displayed on the display, a message stating “good job your blood pressure is improving, you now have a blood pressure of 90” may be displayed. Various embodiments may provide placebo information that encourages the user of the wearable device to continue performing an action that should reduce blood pressure if continued over time. Sensed data may also be shared with the recommendations network 120 and with the mobile device 110. In certain instances, recommendations may be downloaded to the mobile device 110 and recommendations may be displayed on a display at the wearable device. Over time, doctors 170 or trainers 180 may modify recommendation information sent to a wearable device 110. The wearable device may communicate over the cloud or Internet 130 or over optional communication path 150.

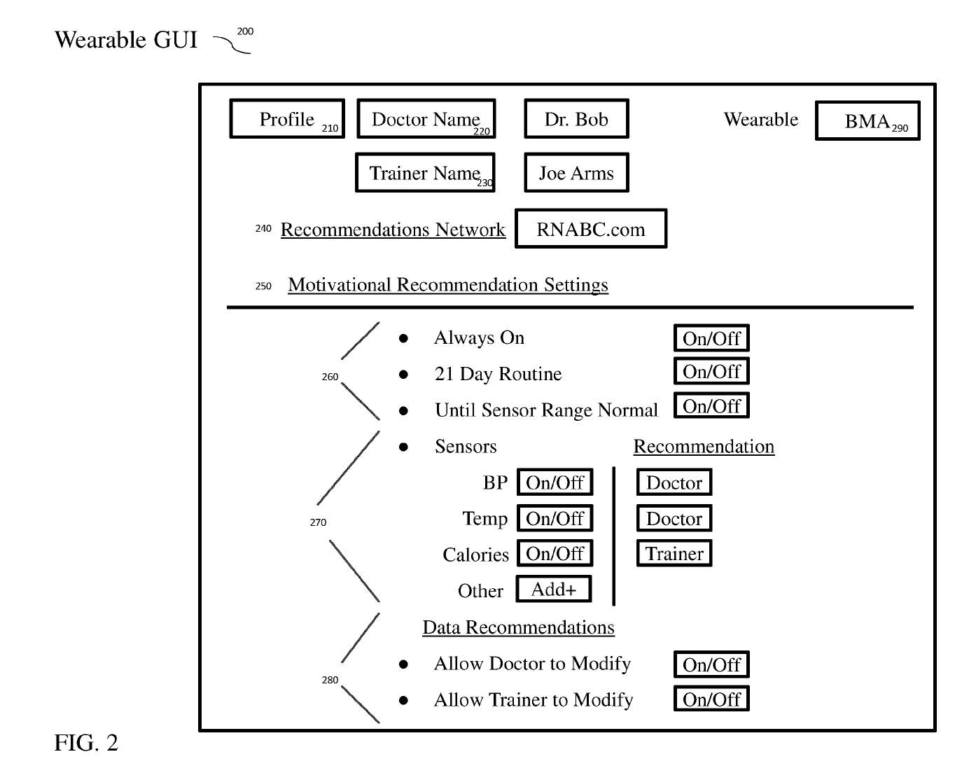

FIG. 2 illustrates an exemplary wearable GUI that may be displayed on the display of the wearable device. The wearable GUI 200 includes a plurality of selection boxes and text entry boxes. The selection boxes include a profile selection box 210 and a plurality of on/off selection boxes 260, 270, & 280. When the profile selection box 210 is selected a user of the wearable device may enter information into a profile GUI at the wearable device. Profile information may include a name of a user, the age of the user, a date, the weight of the user, the height of the user, the sex of the user, or other information. The on/off selection boxes 260, 270, and, 280 illustrated in FIG. 2 enable or disable various functions, features, or a plurality types of physiological data sensed by the corresponding sensors in the wearable device. The feature/function on/off selection boxes 260 illustrated in FIG. 2 include a “21 day routine” option, an “always on” option, t, and a “until sensor range normal” option. The sensor on/off selection boxes 270 include a “blood pressure data” option sensed by a blood pressure sensor, a “temperature data” option sensed by a temperature sensor, a “calorie data” sensed by a calorie sensor, and “another data” sensed by another type of sensor. The sensor selection options 270 allow for switching on or off of the indicated sensor, as well as associated with placebo information. FIG. 2 illustrates that a blood pressure (BP) sensor may be turned on or off in the wearable GUI 200 and that the BP sensor data may be linked to recommendations from a doctor. Similarly, FIG. 2 illustrates that a temperature sensor may be turned on or off in the wearable GUI 200 and shows that the temperature sensor data is linked to recommendations from a doctor. FIG. 2 also depicts that a calorie sensor may be turned on or off in the wearable GUI 200 and that the data from the calorie sensor may be linked to a trainer. Two other on/off selection boxes 280 included in FIG. 2 allow doctor or a trainer to modify or adjust information that may be sent in a message to a wearable device or a user mobile device. The information modified or adjusted may be placebo information that skews, biases, or otherwise alters health information sent to the wearable device or the user mobile device. Data entry boxes in FIG. 2 include a doctor name 220 of Dr. Bob, a trainer name 230 of Joe Arms, a wearable device type provided by vendor Body Media Armband (BMA) 290, and a recommendation network RNABC.com. By using these data entry boxes a user of the wearable may identify a doctor, a trainer, a wearable device type, and a recommendation network.

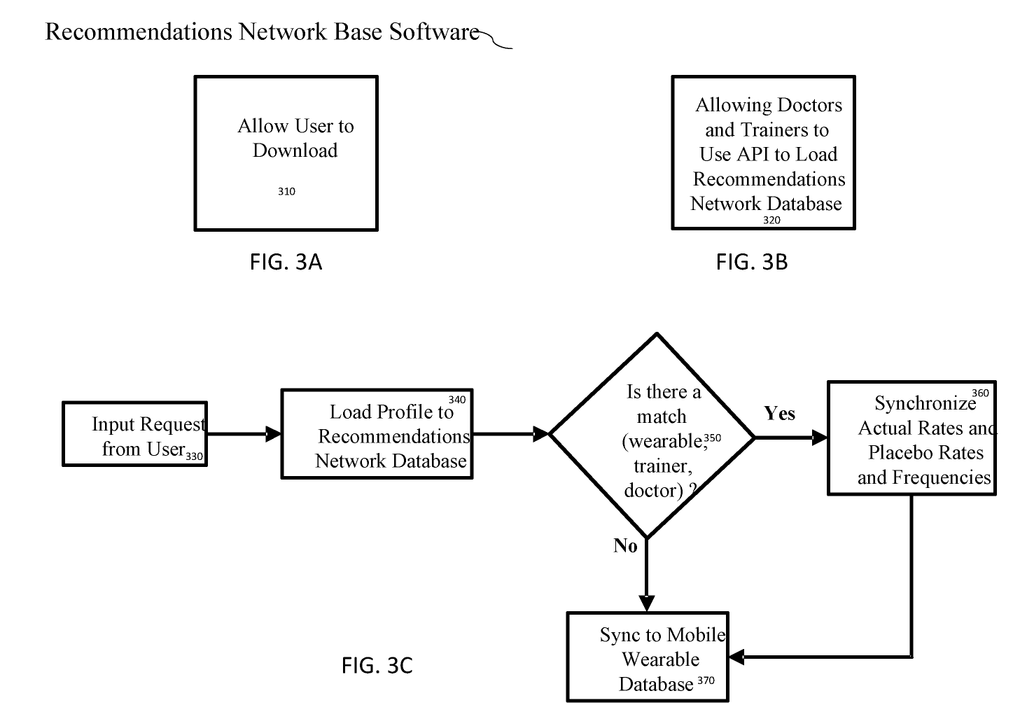

FIGS. 3A-C illustrates an exemplary recommendation network base software that may execute on a recommendation network. In various alternative embodiments, various functions described herein as being performed by the recommendation network may, instead, be performed by another device such as the wearable device itself; modifications for adaptation of the presently-described software to such other devices will be apparent. FIG. 3A illustrates step 310, which allows a user to download information or software from a recommendation network 310. FIG. 3B indicates step 320, which allows doctors and trainers to use an API when uploading information into a recommendation network database at the recommendation network.

FIG. 3C illustrates an exemplary program flow of the recommendations network base. In a first step 330 of FIG. 3C, an input request is received from a user. The input request may be input by a user over a GUI at a wearable device or at a user mobile device. In a second step 340 of FIG. 3C, a user may enter a profile information into the recommendation network database. Profile information may include a name of a user, the age of the user, a date, the weight of the user, the height of the user, the sex of the user, or other information. A third step 350 of FIG. 3C determines whether there is a match between the wearable a trainer or a doctor. When there is no match between the wearable and a trainer or doctor program flow may move to step 370, where the wearable device may synchronize information with the mobile wearable database on a user mobile device. Information that may be synchronized includes, yet is not limited to actual rates, placebo rates, and a frequency. Actual rates may be actual measurements or a range of actual measurements made at the wearable device. An example of an actual rate is a blood pressure measurement. Placebo rates are rates that may be reported to a user of a wearable device that may be biased, skewed, or offset from the actual measurements made at the wearable device. A frequency may relate to a time interval when a wearable device will record sensor data or may relate to a sample rate that corresponds to how frequently data from a particular sensor is sensed.

When there is a match between the wearable device that the trainer or a doctor program flow may move to step 360, where the wearable device may synchronize actual rates and placebo rates and frequencies with information from the recommendation network. After step 360, program flow may move to step 370. the wearable device is allowed to synchronize information with the user mobile device wearable database.

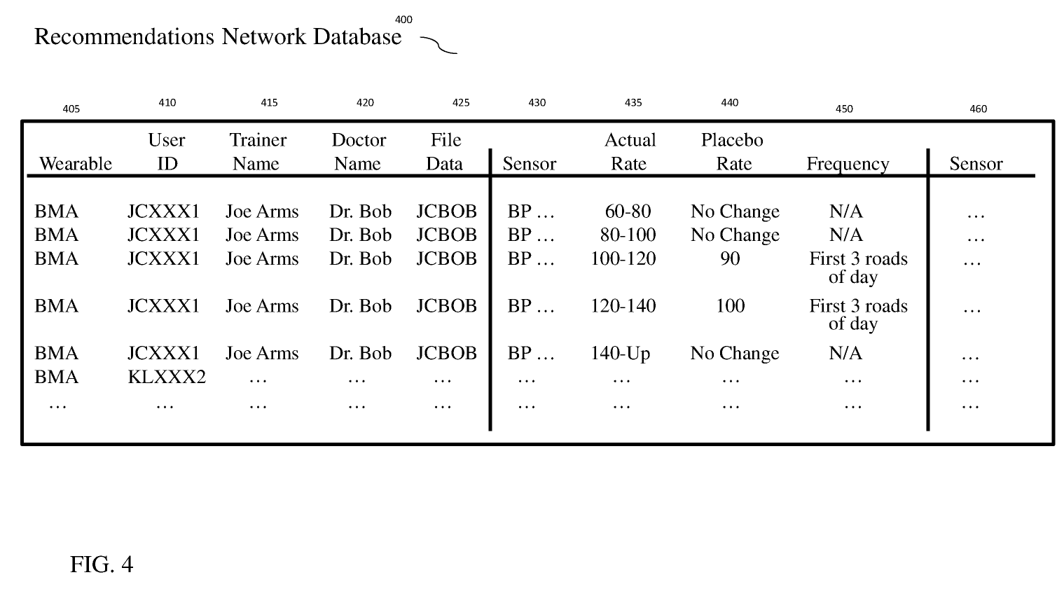

FIG. 4 illustrates a table that cross-references data that may be stored in a wearable database. FIG. 4 illustrates a table 400 that cross-references a wearable device type 405, with a user ID 410, with a trainer name 415, with a Dr. name 420, with file data 425, with specific sensors 430, with actual rate data 435, with a placebo rate 440, and with a frequency 450. The table 400 also includes a column where sensors may be identified 460, this column currently does not identify any particular sensor. The wearable device type identified in each row FIG. 4 is “BMA.” The BMA device is used by user ID JCXXX1. These table entries cross references information that may be communicated with trainer Joe arms or with Dr. Bob.

File data 425 in the first row of the table of FIG. 4 is identified by the file name of JCBOB. This first row of data also includes sensor data of actual blood pressure BP rates where an actual BP rate varies between 60 and 80. The placebo rate 440 in the first row of the table indicates that the blood pressure placebo rate in column 440, first row has not changed. The placebo rates 440 illustrated in FIG. 4, thus, may not directly correspond to the actual BP rates measured by a sensor at the wearable device.

In another example, the third row of the table indicates that a user blood pressure varies between 100 and 120, the placebo rate in column 440, third row in the table that may be reported to a user is 90. Since the placebo rate in column 440, third row of 90 corresponds to an improved blood pressure rate as compared to a range of 100 to 120, a user of the wearable device will be encouraged to continue wearing their wearable device.

The placebo rate provided to a user of a wearable device may be altered in a positive direction (e.g., downward for blood pressure or upward for heart rate during or immediately following exercise) encouraging the user to continue wearing their wearable device. Alternatively, in some embodiments, the placebo alteration may be provided in a negative direction (e.g. downward for calories burned) where such alteration may be deemed motivating to the user. Different users may be affected differently by the same type of placebo alteration. In some embodiments, one or more training algorithms may be applied to each individual user to gauge the results of various types, directions, and parameters for placebo alteration. For example, during a training phase or periodically during normal operation, the wearable device may apply a placebo alteration and record the results from the user (e.g., change in one or more physiological parameters in the time period following the application of the placebo alteration) as a training example. Then, a machine learning method, such as logistic regression, may be applied to a set of training examples to identify the most efficacious placebo alterations for the user. In some such embodiments, the user's current context (e.g., physiological parameters prior to application of the placebo alteration) may form part of each training example and, as such, the trained model may also take into account the user's current context when selecting an appropriate placebo alteration.

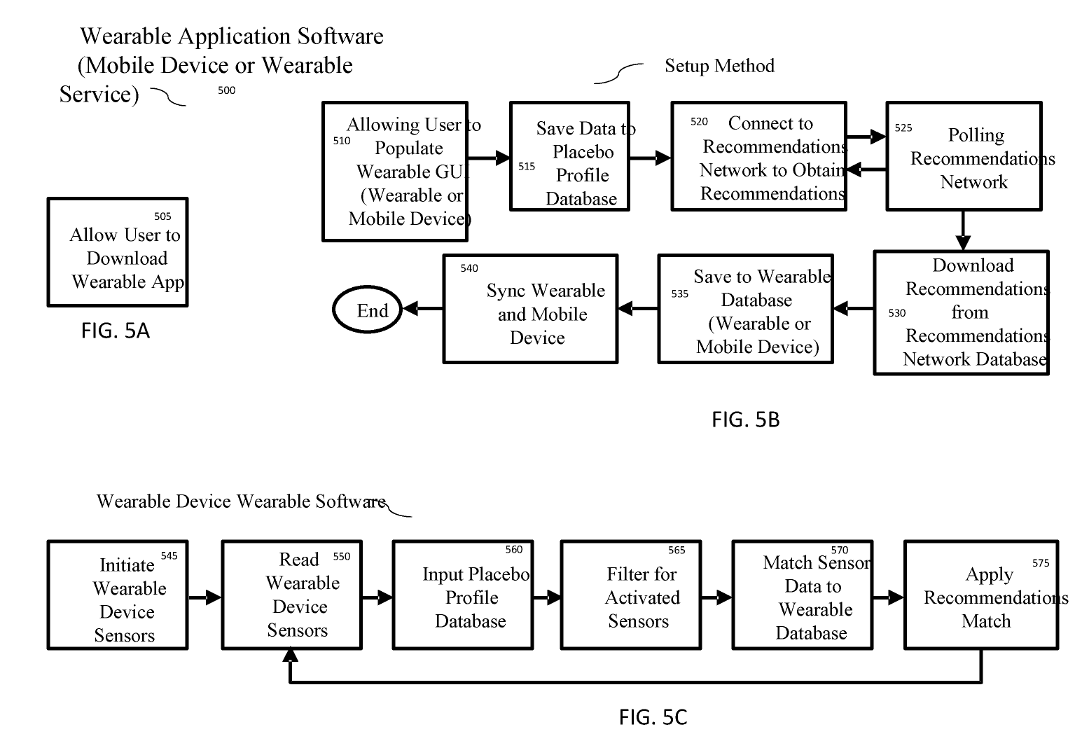

FIG. 5 illustrates an exemplary implementation of wearable application software and wearable software running on a wearable device. FIG. 5A illustrates where a user is allowed to download a wearable application 505. The wearable application software illustrated in FIG. 5A may be running a mobile device or on a wearable device as a service. FIG. 5B begins with step 510 of the wearable application software downloaded in step 505 of FIG. 5A. A user is allowed to populate a wearable GUI on the wearable device or a mobile device with profile or other information. In a second step 515 of the wearable application software of FIG. 5B, the data entered by the user into the GUI is saved in a placebo profile database. Next in a third step 520, the wearable device or the mobile device is allowed to connect to a recommendation network. In this third step 520, the wearable device or the mobile device may be allowed to obtain recommendation information from the recommendation network. Then in a fourth step 525 of FIG. 5B, the recommendation network may be polled for additional information.

The fifth step 530 of FIG. 5B of the wearable application software may download a recommendation from the recommendation network database. The downloaded recommendation may then be saved in a wearable database at the wearable device or at the mobile device in step 535. Then in a seventh step 540 of the wearable application software of FIG. 5B, the wearable device or the mobile device may synchronize information with each other or with the recommendation network.

FIG. 5C illustrates steps that may be performed by wearable software operating on a wearable device. The wearable software method of FIG. 5C begins with a first step 545, where sensors at the wearable device may be initiated or turned on. In a second step 550, wearable device sensor data may be read by or input into the wearable device. Then in a third step 560, user profile information may be input or stored in a placebo database. In a fourth step 565, the activated sensors may be filtered such that information sensed by sensors at the wearable device are limited to sensor data from only the activated sensors. In a fifth step 570 of FIG. 5C, the wearable software sensor data is matched to data in a wearable database. Then in a sixth step 575 of the wearable software, a recommendation corresponding to the match may be used (applied) by the wearable software when preparing a communication with a recommendation network. After the sixth step 575 of FIG. 5C, the wearable software program flow moves back to the second step 550.

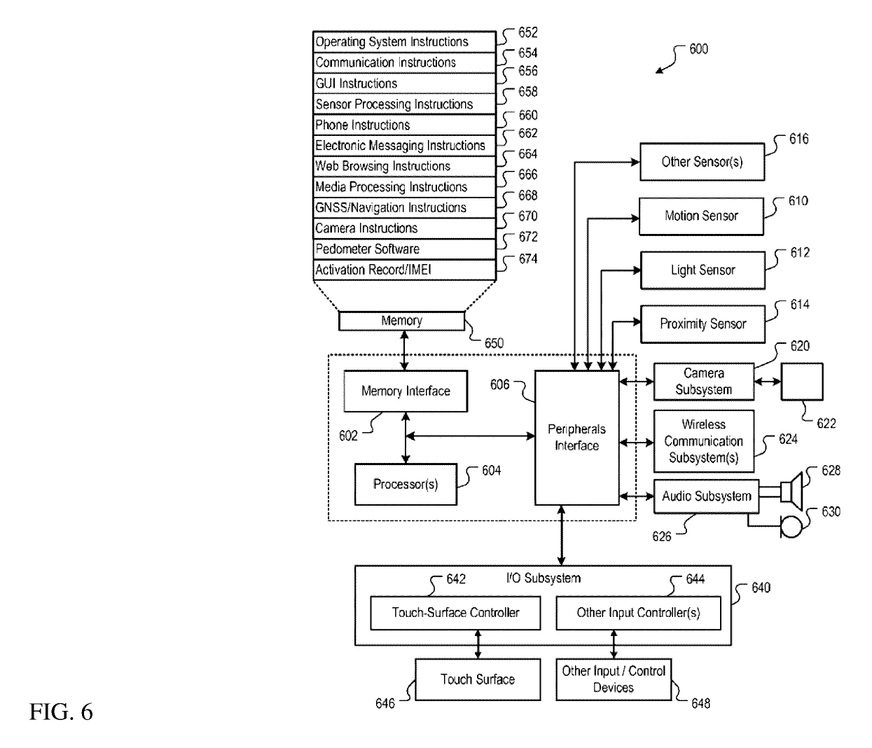

FIG. 6 illustrates a mobile device architecture that may be utilized to implement the various features and processes described herein. Architecture 600 can be implemented in any number of portable devices including but not limited to smart wearable devices. Architecture 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 are meant to be inclusive of data processors, image processors, central processing unit, or any variety of multi-core processing devices. Any variety of sensors, external devices, and external subsystems can be coupled to peripherals interface 606 to facilitate any number of functionalities within the architecture 600 of the exemplar mobile device. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 606, such as a temperature sensor, a biometric sensor, or other sensing device to facilitate corresponding functionalities. Location processor 615 (e.g., a global positioning transceiver) can be coupled to peripherals interface 606 to allow for generation of geo-location data thereby facilitating geo-positioning. An electronic magnetometer 616 such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips. Communication functionality can be facilitated through one or more communication subsystems 624, which may include one or more wireless communication subsystems. Wireless communication subsystems 624 can include 802.5 or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.5 communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions. I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644 can be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, or WINDOWS operating systems, or an embedded operating system such as VXWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; and instructions 672 for any other application that may be operating on or in conjunction with the mobile computing device. Memory 650 may also store other software instructions for facilitating other processes, features and applications, such as applications related to navigation, social networking, location-based services or map displays.

In some embodiments, recommendations may also be provided when drugs or placebos drugs are provided to a user of the wearable device. In such an instances, a user of the wearable device after consuming a placebo drug may be provided with messages that indicate that the medication is providing a desired benefit to the user of the wearable device. Information provided in these messages may include data that is skewed, biased, or otherwise altered. Effects of the user consuming a placebo drug and receiving messages that include biased data may have a double placebo effect that causes the user to relax that in turn helps reduce the user's blood pressure. In instances where actual drugs are administered, messages provided to the user of the wearable device may also be biased and may also cause the user to relax, which in turn may help lower the user's blood pressure.

In other instances, a user of a wearable device may be administered a placebo drug in a first series of administrations and receive a real drug in a second series of administrations. The wearable device may be aware of when the placebo drug was administered and when the real drug was administered. Here again, messages provided to the user of the wearable device may include biased placebo information. In these instances, administration data and sensor data may be sent to the recommendations network where they may be analyzed by a doctor. Administration data may identify when a patient received a placebo drug and when the patient received a real drug. The doctor reviewing the administration data and the sensor data may then determine one or more factors that best work for the user of the wearable device. Certain individuals may be found to receive significant measureable benefits from the placebo drug or from receiving biased placebo information. Other individuals may be found to be influenced more by one type of placebo than another.

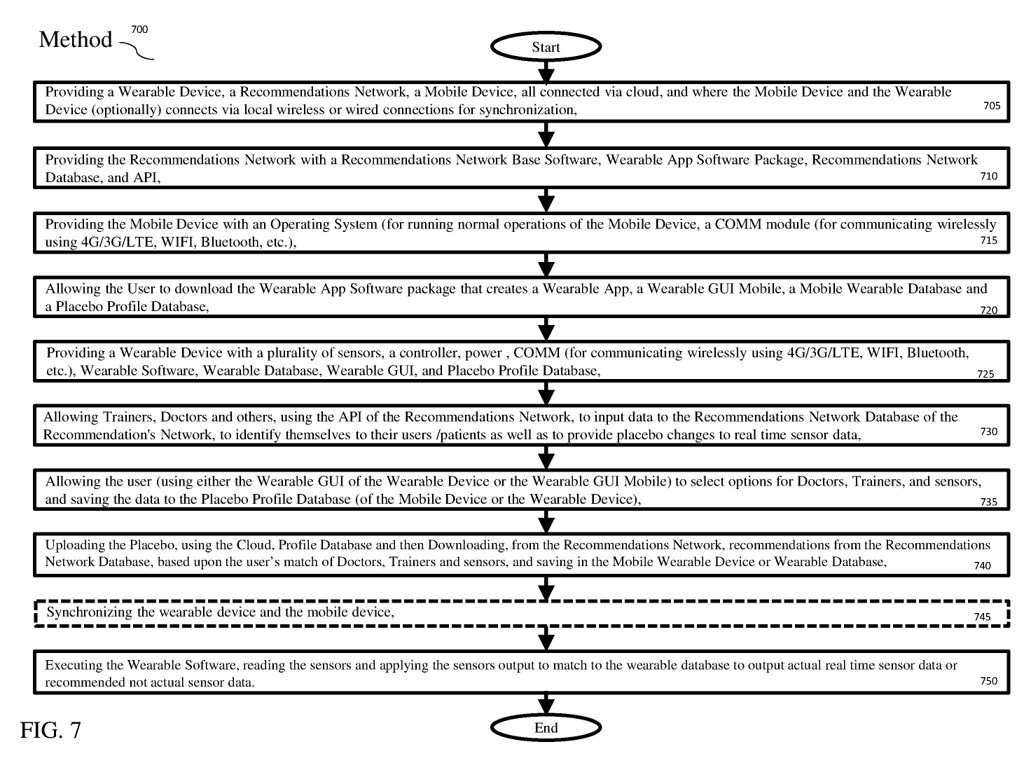

FIG. 7 illustrates an exemplary method that may be implemented according to an embodiment of the invention. In a first step 705 of FIG. 7, a wearable device, a recommendation network, and a mobile device are provided with the ability to communicate wirelessly. These devices may communicate over the cloud or over any other wireless or wireline communications technology known in the art. In a second step 710 of the method of FIG. 7 the recommendation network is provided with recommendation network base software, a wearable application software package, a recommendation network database, and an application program interface API.

In a third step 715 of the method of FIG. 7, the mobile device is provided with operating systems software. In step 715 the mobile device may also be provided with a communication interface that may over any standard wireless technology known in the art including, yet not limited to 3G, 4G, LTE, Wi-Fi (802.X.X), or Bluetooth.

In step 720 of the flowchart of FIG. 7, a user is allowed to download a wearable application software package. The wearable application software package may allow user to create a wearable application, communicate using a wearable GUI, to configure a mobile wearable database, or to configure a placebo profile database.

Then in step 725 of the flowchart of FIG. 7, a wearable device is provided with a plurality of sensors, a controller, a power supply, a communication interface, wearable software, a wearable database, a wearable GUI, and a placebo profile database. In next step 730 of the flowchart of FIG. 7, a trainers, doctors, and others are allowed to enter information using an application program interface at the recommendations network database of the recommendations network. Then in step 735, the user may select options for doctors, trainers, and select which sensors to activate into a GUI at the wearable device. The user may also save the data entered into the placebo profile database located at the mobile device or at the wearable device in this step.

Then in an eighth step 740, profile database information may be uploaded to the recommendation network. The recommendation information may be downloaded from the recommendation network or from the recommendation network database. In certain instances information will be downloaded from the recommendation network database according to a match corresponding to doctors, trainers, and/or sensor data. In this step the information received from the recommendation network database may be stored at the mobile wearable device or in a wearable database at the mobile device.

A ninth step 745 in the FIG. 7 is an optional step where wearable device and a mobile device may synchronize information. Finally, in step 750 of the method of FIG. 7, wearable software on the wearable device may be executed such that sensor data may be sensed. This step may also review the sensor data when matching information stored in the wearable database. This step may also review real-time sensor data, evaluate other information, and may output the real-time sensor data or other information from the wearable device.

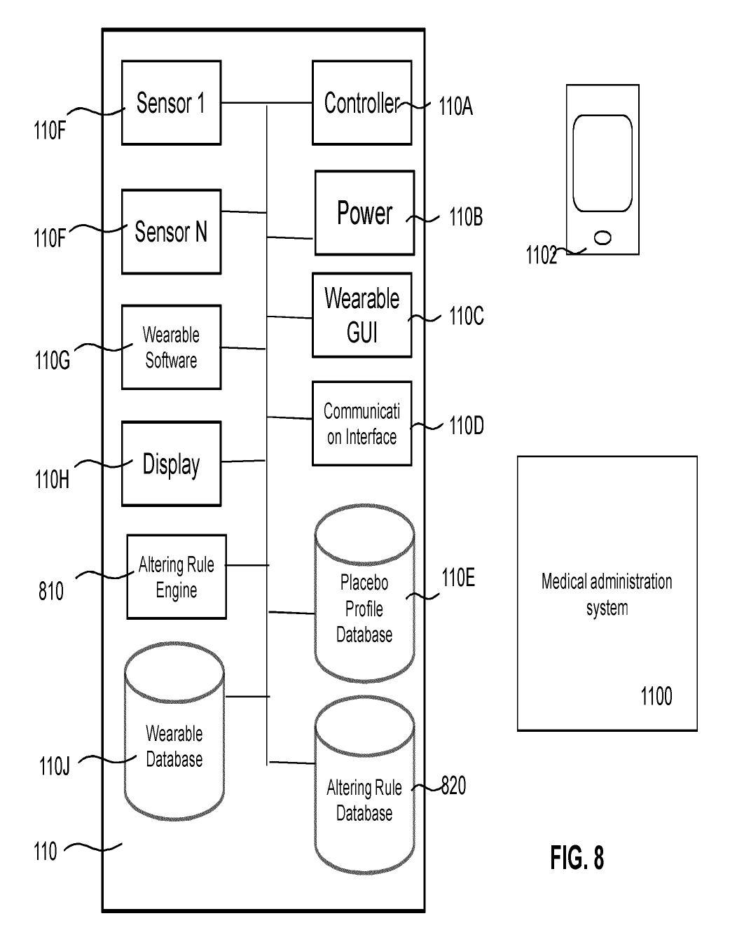

FIG. 8 shows that the wearable device 110 may further comprise an altering rule engine 810 for altering the physiological data sensed from the user of the wearable device 110, and an altering rule database 820. The altering rule database 820 stores a data structure table 830 that may correspond to an altering rule 840 for a specific wearer. The communication interface 110D may communicate with at least one other device, such as a medicine administration system 1100, and/or another wearable device or mobile device 1102.

The wearable graphical user interface (GUI) 110C may receive user's input, such as a user ID.

The communication interface 110D may receive data from the medicine administration system 1100 indicating the medicine that the user of the wearable device 110 may take. Such medicine may include real medicine or placebo medicine. The communication interface 110D may also send suggestion information to the medicine administration system 100 to recommend whether the real medicine or placebo medicine need to be distributed to the user of the wearable device 110 as well as the amount of such medicine that the user may need to take. The communication interface 110D may send information, such as the user ID and the sensed physiological data of the user, to another mobile device 1102 that may be used by a medical professional or trainer. The communication interface 110D may also receive data from the mobile device 1102 including, but not limit to, the medical professional's input, such as the type of placebo information, and/or the value of the placebo information to be provided to the user. The communication interface 110D may further allows the medical professionals or trainers to modify the data structure table 830, such as the frequency rate.

The data structure table 830 may store various rules for placebo alteration which may be received from, for example, a recommendation network. Various alternative approaches to defining placebo alteration rules will be apparent such as, for example, applying a learned model (e.g. learned via linear regression) to various input parameters (including the parameter to be altered).

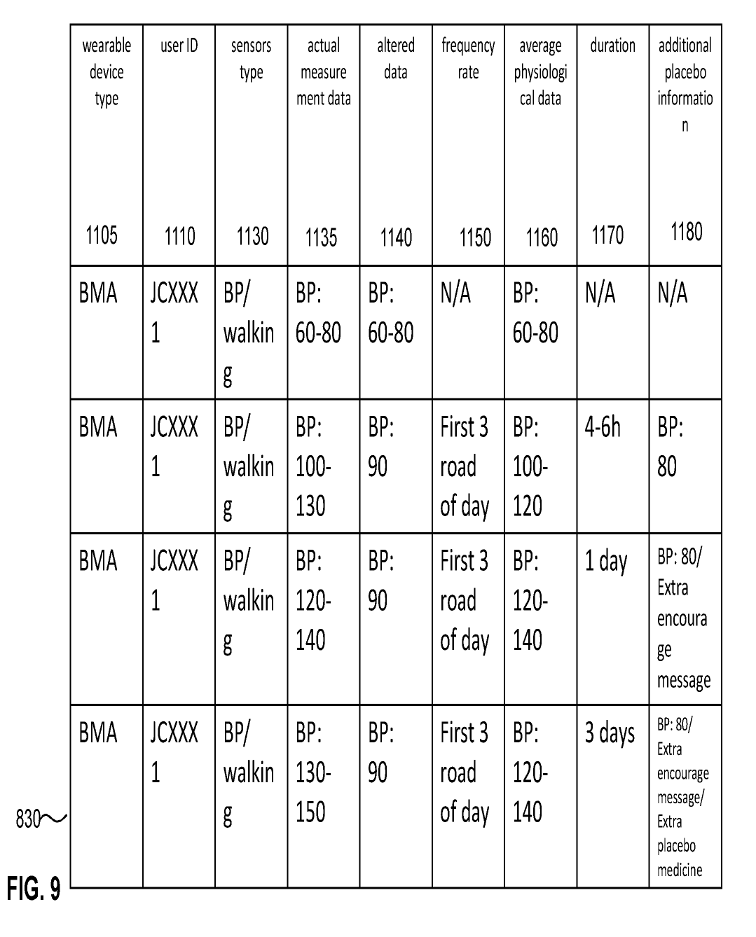

FIG. 9 shows a data structure table 830 that may be stored in an altering rules database 820. The data structure table 830 include a wearable device type field 1105 for indicating the type of the wearable device; a user ID field 1110 for indicating the identification of the current user; sensors type field 1130 for indicating the types of the sensor such as motion sensor, e.g. accelerometers, for detecting the current activity of the user, and/or a physiological measurement sensor, such as a non-invasive blood pressure sensor, for measuring the current blood pressure of the user; an actual measurement data field 1135 for indicating the accurate physiological data sensed by the one or more sensors 110F of the wearable device 110; an altered data field 1140 showing a first altered physiological data indicating the inaccurate physiological data, such as a placebo data, that is intended to be sent to the user; a frequency rate field 1150 indicating the frequency that physiological data need to be sensed; an average physiological data field 1160 indicating the average physiological data of the user sensed by the wearable device 110 during a certain period of time, such as over a few minutes; a duration field 1170 indicating the duration that the inaccurate physiological data is provided to the user; and an additional placebo information field 1180 for defining one or more additional placebo information to be sent to the user. The first altered physiological data may be lower than the actual sensed physiological data, e.g. for around 10-20 BP rates. The one or more additional placebo information may include a second altered physiological data which is even lower than the first altered physiological data, e.g. for around 20-40 BP rates lower than the actual sensed physiological data. Alternatively, the one or more additional placebo information may include information to be sent to the medicine administration system 1100 for indicating an increase of the amount of placebo medicine to the current user. Alternatively, the one or more additional placebo information may include additional encourage information to the user.

As an example, as shown in the second record of the data structure 830, when an a blood pressure of a user while walking is measured to be between 100-130 and the user has been averaging a blood pressure between 100-120, the output blood pressure will be altered down to a value of 90. It will be apparent that various alternative methods of defining an alteration to a physiological parameter will be apparent. In some embodiments, rather than specifying a constant altered value, a rule may specify a mathematical adjustment to the measured value. For example, a rule may indicate that, when the rule is applicable, the measured value should be reduced by a set amount (e.g., 20), increased by a set amount, multiplied by a set factor, divided by a set factor, or modified according to some other mathematical operation or combination thereof (e.g., by specifying a mathematical formula taking into account the measured parameter, constants, coefficients, or variables such as demographics and other physiological parameters).

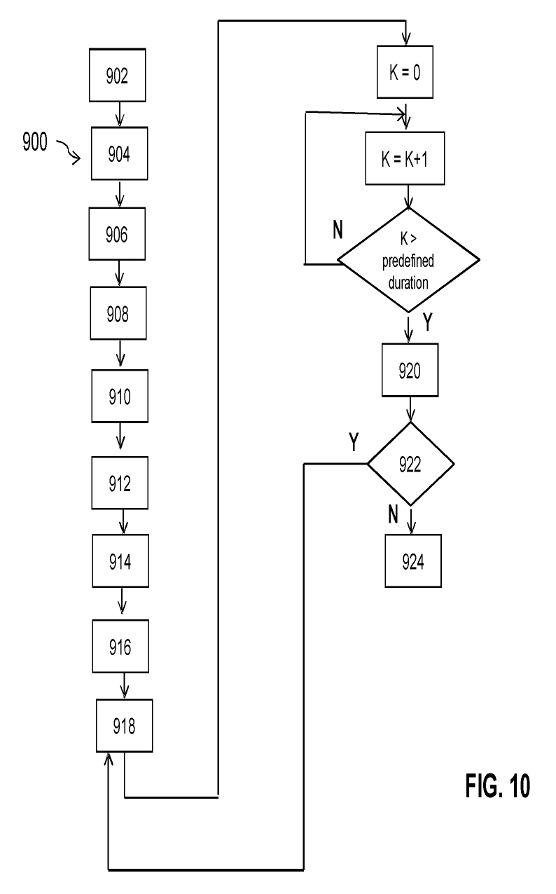

FIG. 10 shows an example of a method 900 performed by the altering rule engine 810 for altering the physiological data sensed by the wearable device 110. The method 900 may correspond to the altering rule database 820 be executed, for example, periodically based on a predefined duration or upon receiving new parameters, e.g., sensor data derived from a particular wearer or data received from the user or medical professionals.

The method 900 begins in step 902 and proceeds to step 904 where the altering rule engine 810 retrieves a user profile for the wearer currently being evaluated based on the current user ID data provided by the user. Based on the user profile, the altering rules engine 810 identifies relevant sensors for the wearer and retrieving sensor data from the identified sensors in step 906. For example, the altering rules engine 810 may extract all available sensors or may extract only those sensor implicated by the data structure table 830 such as the sensors for indicating the user activity and/or the sensors for sensing a certain type of physiological data. Next, in step 908, the altering rule engine 810 may obtain any new physiological data available from the sensors of wearable device 110. Next, in step 910, the altering rule engine 810 calculates an average physiological data based on both the previously retrieved physiological data and the new retrieved physiological data. In step 912, the altering rule engine 810 obtains data structure table 830. In step 914, the altering rule engine 810 determines the current user activity based on the data sensed from, e.g. the motion sensor. In step 916, the altering rule engine 810 determines a first altered physiological data to be provided to the user based on the altered physiological data in the data structure table 830 corresponding to the calculated average physiological data and the determined user activity. In step 918, the altering rule engine 810 transmits the first altered physiological data to the communication interface 110D in order to the sent to the user for a predetermined duration as defined in the data structure table 830.

Note that, in various alternative embodiments, the method 900 need not or otherwise does not take steps to obtain sensor data or other parameters. For example, in some embodiments, the wearable device 110 may periodically store extracted parameters and new sensor data in a location accessible to the altering rule engine 810 such as, for example, the user profile. As such, in some embodiments, one or more of the steps for obtaining parameters may be omitted or modified to simply read the values from the expected location. In addition, in various alternative embodiments, the altering rule engine 810 may determines the first altered physiological data to be provided to the user solely based on the calculated average physiological data and without taking into account the user activity in step 916. Likewise, there may be no need to derive the sensor data related to the user activity in step 906. Furthermore, the reason of calculating the average physiological data based on both the previously retrieved physiological data and the new retrieved physiological data within a few minutes is to derive a reliable and stable physiological data for determining the current physiological status of the user. It is understood that the step 910 of calculating the average physiological data can be omitted if the user is in a stable mode, e.g. walking or lying on bed. Instead, in step 916, the altering rule engine 810 may determine a first altered physiological data to be provided to the user based on the altered physiological data in the data structure table 830 corresponding to the currently sensed actual physiological data (and the determined user activity).

In step 920, after a duration that the first altered physiological data is provided to the user, e.g. 4-6 hours, as defined in the data structure table 830, the altering rule engine 810 may further recalculate the average physiological data based on both the previously retrieved physiological data and the new retrieved physiological data within a few minutes, e.g. 3-5 minutes. This recalculated average physiological data may reflect the reaction of the user to the first altered physiological data. In step 922, the altering rule engine 810 compares whether the difference between the recalculated average physiological data and the first altered physiological data is within a certain range, e.g. 5-10 BP rates. If the difference is within the certain range, the altering rule engine 810 will continue transmitting the first altered physiological data to the communication interface 110D. If the difference is larger than certain range, depending on the duration of the first altered physiological data being provided to the user and/or the value of the difference, the altering rule engine 810 may provide different types of placebo information to the user based on the data structure table 830 in step 924. For instance, if the duration that the first altered physiological data being provided to the user is more than one day and/or the value difference is more than 20 BP rates, the altering rule engine 810 may send a second altered physiological data, e.g. 80 BP rates, which is even lower than the first altered physiological data to the communication interface 110D. If the duration that the first altered physiological data being provided to the user is more than three days, an/or the value difference is more than 20-30 BP rates, the altering rule engine 810 may send the second altered physiological data together with additional encouraging information to communication interface 110D which will be shown to the user. Alternatively, the altering rule engine 810 may communicate with the medical administration system to suggest delivering extra placebo medicine to the user. As already discussed above, the reason of recalculating the average physiological data based on both the previously retrieved physiological data and the new retrieved physiological data within a few minutes is to derive a reliable and stable physiological data for determining the current physiological status of the user. It is understood that the step 920 of recalculating the average physiological data can be omitted if the user is in a stable mode, e.g. walking lying on bed. Instead, in step 922, the altering rule engine 810 may only need to compare whether the difference between the currently sensed actual physiological data and the first altered physiological data is within a certain range or not.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A computer-implemented method for altering sensed physiological data derived from a user of a wearable device, the method comprising:
receiving one or more types of physiological data of the user sensed by one or more corresponding sensors in the wearable device;
altering a value of the one or more types of physiological data to a first altered physiological data based on a predetermined altering rule database while keeping the types of physiological data unchanged;
notifying said user regarding the first altered physiological data.

2. The computer-implemented method of claim 1, wherein the altered physiological data is an inaccurate physiological data with respect to the physiological data sensed by the corresponding sensors in the wearable device.

3. The computer-implemented method of claim 1, wherein the altered physiological data includes placebo data.

4. The computer-implemented method of claim 1, further comprising:
detecting the reaction of the user in response to the first altered sensed physiological data;
and providing different types of placebo information to the user in response to the detection result.

5. The computer-implemented method of claim 4, wherein detecting the reaction of the user in response to the first altered sensed physiological data further comprising the steps of:
receiving one or more types of physiological data of the user sensed by one or more corresponding sensors in the wearable device after a predefined duration that the first altered physiological data is provided to the user;
comparing the difference of the received physiological data of the user and the first altered physiological data is provided to the user.

6. The computer-implemented method of claim 4, wherein the different types of placebo information comprise at least one of:
a second altered physiological data; and
additional encouraging information.

7. The computer-implemented method of claim 4, further comprising
communicating with a medicine administration system by indicating whether the placebo medicine needs to be provided to the user, or whether an increase of the amount of placebo medicine to the current user.

8. Computer-implemented method of claim 1, wherein the predetermined altering rule database is stored in a recommendation network.

9. Computer-implemented method of claim 8, further comprising:
transmitting the physiological data sensed by the one or more sensors to a recommendation network;
receiving an alternated physiological data from the recommendation network,
notifying the received alternated physiological data to the user.

10. Computer-implemented method of claim 1, further comprising receiving a selection of a professional that may receive the physiological data sensed at the wearable device.

11. Computer-implemented method of claim 10, further comprising receiving an indication allowing the professional to modify the physiological data sensed by the one or more corresponding sensors, wherein a modification by the professional skews physiological data sensed by the one or more corresponding sensors, the modified physiological data corresponds to the predetermined physiological data.

12. A wearable device comprising:
a communication interface configured to receive data from at least one other device and to send a notification to a user of the wearable device or information to at least the one other device,
a processor configured to perform a method according to claim 1.

13. The wearable device according to claim 13, further comprising: —a memory configured to store a predetermined altering rule database.

14. An altering rule engine 810 for altering physiological data sensed from a user of a wearable device, comprising:
a communication interface configured to communicate with at least one other device and to send a notification to the user of the wearable device or information to at least the one other device,
a processor configured to perform a method according to claim 1.

15. A non-transitory computer readable data storage medium having embodied thereon a program executed by a processor to perform a method according to claim 1.
